(19) United States
(12) Patent Application Publication
(10) Pub. No.: US 2024/0400601 A1
(43) Pub. Date: Dec. 5, 2024

(54) PROCESS FOR THE PREPARATION OF LOW-COLOR ALKYL POLYGLYCOSIDES, INVOLVING NEUTRALIZATION OF THE REACTION MEDIUM BEFORE REMOVAL OF THE SUGAR

(71) Applicant: SOCIÉTÉ D'EXPLOITATION DE PRODUITS POUR LES INDUSTRIES CHIMIQUES SEPPIC, PARIS CEDEX 07 (FR)

(72) Inventors: Estelle ILLOUS, CASTRES (FR); Stéphane DESSILLA, CASTRES (FR)

(21) Appl. No.: 18/700,569

(22) PCT Filed: Oct. 12, 2022
(86) PCT No.: PCT/EP2022/078397

§ 371 Date: Apr. 11, 2024

(30) Foreign Application Priority Data
Oct. 13, 2021 (FR) FR2110842

(51) Int. Cl. C07H 15/04; C07H 1/00

(52) U.S. Cl. CPC C07H 15/04; C07H 1/00

(57) ABSTRACT

Disclosed is a process for preparing alkyl polyglycosides having a color of less than or equal to 1.5 VCS, the process successively involving: a) a glycosylation step a) consisting in a reaction between at least one alcohol of formula (I) and at least one reducing sugar of formula (III): H—O-(G)-H (III), in the presence of at least one acid catalyst (CA) at a minimum temperature of 100° C. and a maximum temperature of 120° C., b) a step b) of neutralizing the reaction medium from step a) with an aqueous solution comprising a basic agent (Ab), c) a step c) of removing the sugar of formula (III), which has not reacted in step a), from the neutralized reaction medium, and d) a step d) of recovering at least one composition (C) having a color of less than or equal to 1.5 VCS.

The present invention relates to a process for preparing low-color alkyl polyglycosides (color of less than 1.5 VCS) involving carbonate neutralizing agents.

Alkyl polyglycosides or APGs are probably the best examples of biobased surfactants available on the market today. Their molecular structures are characterized by the simultaneous presence of a hydrophilic head derived from reducing sugars (D-glucose, D-xylose or D-rhamnose are the reducing sugars mainly available on the market on an industrial scale) and a lipophilic hydrocarbon chain of varying length (cf. formula I: simplified structure of an APG).

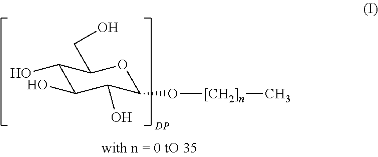

The process for the industrial scale manufacture thereof is relatively simple and uses, as raw materials: i) crystalline glucose, xylose or rhamnose resulting respectively from the complete hydrolysis of wheat, corn or potato starch or from the hydrolysis of wood hemicelluloses; and ii) fatty alcohols from the oleochemical industry (hydrogenation of methyl esters resulting from the transesterification of plant triglycerides). Fischer glycosylation reactions then consist in linking these two raw materials together by creating a covalent chemical bond, like for example in reaction (II) between glucose and an alcohol.

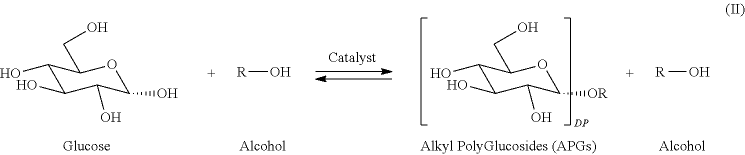

To carry out this glycosylation reaction, an acid catalyst of mineral or organic origin is necessary and an excess of alcohols is systematically introduced, thus acting as reactant and solvent. At the end of the reaction, the APGs are dispersed or dissolved in the excess unreacted alcohol.

APGs are distinguished by the nature and length of the hydrocarbon alkyl chain R and by their average degree of polymerization DP of greater than 1 but less than or equal to 2.5.

At the end of the glycosylation reaction phase, a neutralization step is carried out in order to deactivate the catalyst and stop the reaction.

Depending on the length of the alkyl chain of the alcohol and the associated use, said alcohol is either removed or retained.

The neutralization step differs depending on the length of the hydrocarbon alkyl chain. In the case where the latter has a number of carbon atoms of less than 12, the neutralization is carried out by an aqueous solution of sodium hydroxide. The excess fatty alcohols present at the end of glycosylation are then removed by high vacuum distillation or molecular distillation, or by evaporation, generally using a falling-thin-film evaporator or a short-path thin-film evaporator, and the APG concentrate collected is finally dissolved in water. The commercial products thus obtained are therefore in the form of aqueous APG solutions with a weight concentration of between 40% and 80%.

In the case in which the hydrocarbon alkyl chain R has a number of carbon atoms of greater than or equal to 12, the neutralization is generally carried out by sodium hydroxide or by potassium hydroxide, alone or in combination with a reducing agent such as described in the European patent published under number EP 0 077 167, in the European patent application published under number EP 0 338 151 A1, in the European patent EP 0 388 857 B1, for instance sodium borohydride (NaBH₄) or sodium hypophosphite (NaH₂PO₂). The mixture of APG and excess fatty alcohols is isolated after neutralization and is sold as is. The weight proportion of APGs and fatty alcohols depends on the molar stoichiometry adopted at the start for the raw materials and on their reactivity. However, proportions of from 5% to 30% by weight of APG and from 70% to 95% of fatty alcohols are generally observed. The compositions obtained may be in solid form, for instance in the form of flakes or beads, or in liquid form, depending on the nature of the hydrocarbon alkyl chain R.

However, the step of neutralizing APGs having a hydrocarbon alkyl chain R that contains a number of carbon atoms of greater than or equal to 12 with a base from the prior art (e.g. NaOH, KOH), in order to achieve a pH of a 5 wt % dispersion in water of the neutralized medium of between 5.5 and 7.5, gives rise to significant coloration of the product. For the purposes of the present invention, “measurement of the pH of a 5 wt % dispersion in water” denotes the analytical method for measuring the pH of a dispersion of an APG-based composition according to the provisions of NF EN 1262; said measurement is carried out by potentiometric measurement using a combined pH electrode (aqueous media) and a pH meter.

This coloration may impair the organoleptic qualities of the finished products into which the APG-containing compositions are introduced. For this reason, solutions are provided to minimize the coloration of compositions containing APGs having a hydrocarbon alkyl chain R that contains a number of carbon atoms of greater than or equal to 12. Two solutions known from the prior art are conventionally used to obtain such low-color (<1.5 VCS) compositions based on APGs having a hydrocarbon alkyl chain R that contains a number of carbon atoms of greater than or equal to 12.

For the purposes of the present invention, a “low-color composition” denotes a composition for which the Gardner color scale, as defined by DIN-ISO 4630, is less than or equal to 1.5 VCS. The Gardner color scale is measured using a LICO 200/Dr LANGE (or equivalent) colorimeter that performs light transmission measurements on any medium. Such a colorimeter operates with a halogen lamp corresponding to the standard illuminant C defined by DIN 5033 and with a 2° standard observer. During the measurement, a reference radiation beam compensates for variations in the recorded values due to lamp and temperature differences.

The first solution consists in combining a reducing agent with the base used. Among these reducing agents, mention may be made of sodium borohydride (NaBH₄) or of sodium hypophosphite (NaH₂PO₂). This solution is not entirely satisfactory. Specifically, although very effective in minimizing the coloration of the treated composition, NaBH4 is a reducing agent which is hazardous to handle and to use (corrosive product, release of hydrogen). NaH₂PO₂ is itself not very effective, even if it is introduced at high concentration. The second solution commonly used and described in the prior art for minimizing the color of compositions based on APGs having a hydrocarbon alkyl chain R that contains a number of carbon atoms of greater than or equal to 12 is carrying out a decolorization with hydrogen peroxide (H₂O₂) during the finishing step. Although it is effective, this step is however tedious because it requires the pH of a 5 wt % dispersion in water to be adjusted to between 7.0 and 7.5 while maintaining the oxidizing power of the medium by adding H₂O₂. This step, which is difficult to carry out, can last several hours and therefore significantly increase the production time, reducing productivity.

The technical problem to be solved is therefore that of finding an alternative to the neutralization of compositions based on APGs having a hydrocarbon alkyl chain R that contains a number of carbon atoms of greater than or equal to 12. This alternative must be effective and easy to implement, while guaranteeing a color of less than or equal to 1.5 VCS without carrying out a decolorization step.

One solution of the present invention is a process for preparing a composition (C) having a color of less than or equal to 1.5 VCS, comprising, for 100% of its weight:
(i) an amount of greater than or equal to 40% by weight and less than or equal to 95% by weight of an alcohol of formula (I):
   R—OH (I), wherein R represents a linear or branched, saturated or unsaturated hydrocarbon radical, which may contain at least one hydroxyl function, and containing from 12 to 22 carbon atoms or a mixture of alcohols of formula (I);
ii) an amount of greater than or equal to 5% by weight and less than or equal to 60% by weight of a composition (C1) represented by formula (11):
   R—O-(G)x-H (II), wherein the residue G represents the residue of a reducing sugar, R represents a radical as defined in formula (I) and x, which indicates the average degree of polymerization of the residue G represents a decimal number greater than 1.05 and less than or equal to 2.5, or a mixture of compositions (C1) of formula (II);
it being understood that the sum of the weight proportions of the compounds in composition (C) of formulae (I) and (II) is equal to 100% by weight,
said process successively comprising:
a) a step a) of glycosylation, consisting of a reaction between at least one alcohol of formula (I) and at least one reducing sugar of formula (III): H—O-(G)-H (III), in the presence of at least one acid catalyst (CA), at a temperature of greater than or equal to 100° C. and less than or equal to 120° C., the acid catalyst (CA) being selected from members of the group consisting of sulfuric acid, hydrochloric acid, phosphoric acid, nitric acid, hypophosphorous acid, methanesulfonic acid, para-toluenesulfonic acid, trifluoromethanesulfonic acid and acid ion-exchange resins,
b) a step b) of neutralizing the reaction medium from step a) with an aqueous solution comprising a basic agent (Ab) selected from the members of the group consisting of:
   carbonates of formula (IVa):
      XnCO₃ (IVa), wherein X represents a sodium or potassium atom and n is an integer equal to 2, or X represents a calcium atom or a magnesium atom and n is an integer equal to 1, or
   hydrogen carbonates of formula (IVb):
      Y(HCO₃)m (IVb), wherein Y represents a sodium or potassium atom and m is an integer equal to 1, or Y represents a calcium atom or a magnesium atom and m is an integer equal to 2,
   this neutralization being carried out so as to obtain a reaction medium for which the 5 wt % dispersion of said reaction medium in water has a pH of between 5.5 and 7.5,
c) a step c) of removing the sugar of formula (III), which did not react in step a), from the neutralized reaction medium, and
d) a step d) of recovering at least one composition (C) having a color of less than or equal to 1.5 VCS.

The color scale characterizing the composition (C) prepared according to the process which is the subject of the present invention is the Gardner color scale, as defined by DIN-ISO 463. The Gardner color scale is measured using a LICO 200/Dr LANGE (or equivalent) colorimeter that performs light transmission measurements on any medium. Such a colorimeter operates with a halogen lamp corresponding to the standard illuminant C defined by DIN 5033 and with a 2° standard observer. During the measurement, a reference radiation beam compensates for variations in the recorded values due to lamp and temperature differences. The unit for expressing the Gardner color scale, characterizing the composition (C) prepared according to the process which is the subject of the present invention, is VCS. Depending on the case, the process according to the invention may have one or more of the following characteristics:
said composition (C1) consists of a mixture of compounds represented by formulae (II1), (II2), (II3), (II4) and (II5):

R—O-(G)1-H (II1),

R—O-(G)2-H (II2),

R—O-(G)3-H (II3),

R—O-(G)4-H (II4),

R—O-(G)5-H (II5),
in the respective molar proportions a1, a2, a3, a4 and a5, such that:
   the sum: a1+a2+a3+a4+a5 is equal to 1, and
   the sum a1+2a2+3a3+4a4+5a5 is equal to x;
the composition (C) comprises an amount of less than or equal to 2% by weight of the reducing sugar of formula (III):

H—O-(G)-H (III);
it being understood that the sum of the weight proportions of the compounds of formulae (I), (II) and (II1) in composition (C) is equal to 100% by weight;
the basic agent (Ab) present in the aqueous solution is sodium carbonate of formula (IVa) wherein X represents a sodium atom and n is equal to 2;
the process comprises, after step c), an additional step of adjusting the pH of a 5 wt % dispersion of the reaction medium in water by adding an aqueous solution comprising the basic agent (Ab) as defined above, so as to obtain a value of said pH of between 5.5 and 7.5;
the reducing sugar of formula (III) chosen for the glycosylation of step a) is selected from the members of the group consisting of glucose, xylose, arabinose and rhamnose;
step c) of removing the reducing sugar of formula (II1) is carried out by filtration, centrifugation or settling or by any other solid/liquid separation technique known to those skilled in the art; when this step of removing the unreacted reducing sugar of formula (III) employs filtration, the filtering medium can be filter paper having suitable pore sizes (greater than or equal to 10 micrometers), or else a cellulose filter plate optionally in the presence of at least one filtration adjuvant known to those skilled in the art;
in step b), the aqueous solution of basic agent (Ab) comprises between 10% and 40% by weight of said basic agent (Ab);
step a) comprises the following successive substeps:
i) introducing an alcohol of formula (I) or a mixture of alcohols of formula (I) into a reactor (Re) equipped with a mechanical stirrer and a vacuum device;
ii) heating the alcohol of formula (I) to a temperature of between 80° C. and 90° C. under mechanical stirring;
iii) charging the reducing sugar of formula (II1) into the reactor (Re);
iv) introducing at least one acid catalyst into the reactor (Re);
v) heating, under partial vacuum, the reaction medium from substep iv) present in the reactor (Re) to a temperature of between 100° C. and −110° C. for the duration of the reaction, and
vi) cooling the medium from substep v) to a temperature of between 70° C. and 80° C.;
in formula (I) and/or in formula (II), the radical R is selected from the following radicals: lauryl (or n-dodecyl), myristyl (or n-tetradecyl), n-pentadecyl, cetyl (or n-hexadecyl), n-heptadecyl, stearyl (or n-octadecyl), palmitoleyl (or 9-hexadecenyl), oleyl (or 9-octadecenyl), linoleyl (9,12-octadecadienyl), linolenyl (or 6,9,12-octadecatrienyl), n-nonadecyl, arachidyl (or n-eicosyl), behenyl (or n-docosyl), erucyl (13-docosenyl), or 12-hydroxystearyl;
in formula (I) and/or in formula (II), the radical R is selected from the following radicals: 2-hexyloctyl, 2-hexyldecyl, 2-hexyldodecyl, 2-octyldecyl, 2-octyldodecyl, 2-decyltetradecyl, isostearyl (or 16-methylheptadecyl) or isomyristyl (or 13-methyltridecyl);
during substeps ii) to iv), the reactor (Re) is inerted under nitrogen;
the process comprises, between steps ii) and iii), a vacuum step, preferably at a pressure of less than or equal to 50 millibar;
step vi) is carried out at atmospheric pressure.

The use of carbonates of formula (IVa) or hydrogen carbonates of formula (IVb) does not contribute to increasing the color of the composition (C) (the presence of a reducing agent then not being necessary) while neutralizing a 5 wt % dispersion of the composition (C) to the desired pH (the value of which is between 5.5 and 7.5). The use of a basic agent (Ab) makes it possible to avoid a decolorization step involving the use of a peroxide agent, or the like, since it makes it possible to achieve a color less than or equal to 1.5 VCS.

The term “reducing sugar” in the definition of formula (II) and in the definition of formula (III) denotes saccharide derivatives that do not have in their structures any glycosidic bonds established between an anomeric carbon and the oxygen of an acetal group, as they are defined in the reference publication: “Biochemistry”, Daniel Voet/Judith G. Voet, page 250, John Wiley & Sons, 1990.

The oligomeric structure (G)x present in formula (II) may be in any isomeric form, whether this relates to optical isomerism, geometrical isomerism or regioisomerism; it may also represent a mixture of isomers.

In formula (II) as defined above, the radical R is bonded to G via the anomeric carbon of the saccharide residue, so as to form an acetal function.

According to a particular aspect of the present invention, in the definition of the compounds of formulae (II) and of formula (III), G represents the residue of a reducing sugar selected from glucose, dextrose, sucrose, fructose, idose, gulose, galactose, maltose, isomaltose, maltotriose, lactose, cellobiose, mannose, ribose, xylose, arabinose, lyxose, allose, altrose, rhamnose, dextran or tallose.

According to a particular aspect of the present invention, in the definition of the compounds of formula (II), G represents the residue of a reducing sugar selected from the residues of glucose, xylose, arabinose or rhamnose, and x represents a decimal number greater than or equal to 1.05 and less than or equal to 2.5.

According to an even more particular aspect of the present invention, in the definition of the compounds of formula (II), G represents the residue of a reducing sugar selected from the residues of glucose, xylose, arabinose or rhamnose, and x represents a decimal number greater than or equal to 1.05 and less than or equal to 2.0, and even more particularly greater than or equal to 1.25 and less than or equal to 2.0.

According to another particular aspect of the present invention, the reducing sugar of formula (III) is selected from the members of the group consisting of glucose, dextrose, sucrose, fructose, idose, gulose, galactose, maltose, isomaltose, maltotriose, lactose, cellobiose, mannose, ribose, xylose, arabinose, lyxose, allose, altrose, rhamnose, dextran or tallose.

According to a more particular aspect of the present invention, the reducing sugar of formula (III) is selected from glucose, xylose, arabinose or rhamnose.

The process according to the invention consists in neutralizing the medium at the end of the glycosylation reaction by adding an aqueous solution comprising a basic agent (Ab), in order to achieve a pH of a 5 wt % dispersion in water of said medium of between 5.5 and 7.5. The residual reducing sugar of formula (III) is subsequently removed by filtration and, optionally, an aqueous solution of a basic agent (Ab) can be added anew if the pH of the 5 wt % dispersion in water of the composition (C) is less than 5.5 (finishing step).

According to a particular aspect, one subject of the process is the preparation of a composition (C) having a color of less than or equal to 1.5 VCS, comprising, for 100% of its weight:
from 45% to 55% by weight of a mixture (M1) of alcohols of formula (I) comprising, for 100% of the weight of said mixture (M1), 50% by weight of an alcohol of formula (I) wherein R represents the n-hexadecyl radical and 50% by weight of an alcohol of formula (I) wherein R represents the n-octadecyl radical;
from 45% to 54% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical;
from 0% to 1% by weight of glucose.

According to a particular aspect, one subject of the process is the preparation of a composition (C) having a color of less than or equal to 1.5 VCS, comprising, for 100% of its weight:
from 45% to 55% by weight of a mixture (M′1) of alcohols of formula (I) comprising, for 100% of the weight of said mixture (M′1), 70% by weight of an alcohol of formula (I) wherein R represents the n-hexadecyl radical and 30% by weight of an alcohol of formula (I) wherein R represents the n-octadecyl radical;
from 45% to 54% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical;
from 0% to 1% by weight of glucose.

According to a particular aspect, one subject of the process is the preparation of a composition (C) having a color of less than or equal to 1.5 VCS, comprising, for 100% of its weight:
from 75% to 90% by weight of a mixture (M″1) of alcohols of formula (I) comprising, for 100% of the weight of said mixture (M″1), 50% by weight of an alcohol of formula (I) wherein R represents the n-hexadecyl radical and 50% by weight of an alcohol of formula (I) wherein R represents the n-octadecyl radical;
from 10% to 24% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical;
from 0% to 1% by weight of glucose.

According to a particular aspect, one subject of the process is the preparation of a composition (C) having a color of less than or equal to 1.5 VCS, comprising, for 100% of its weight:
from 75% to 90% by weight of a mixture (M′″1) of alcohols of formula (I) comprising, for 100% of the weight of said mixture (M′″1), 70% by weight of an alcohol of formula (I) wherein R represents the n-hexadecyl radical and 30% by weight of an alcohol of formula (I) wherein R represents the n-octadecyl radical;
from 10% to 24% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical;
from 0% to 1% by weight of glucose.

According to a particular aspect, one subject of the process is the preparation of a composition (C) having a color of less than or equal to 1.5 VCS, comprising, for 100% of its weight:
from 75% to 90% by weight of an alcohol of formula (I) wherein R represents the n-tetradecyl radical;
from 10% to 24% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-tetradecyl radical;
from 0% to 1% by weight of glucose.

According to a particular aspect, one subject of the process is the preparation of a composition (C) having a color of less than or equal to 1.5 VCS, comprising, for 100% of its weight:
from 75% to 90% by weight of a mixture of alcohols of formula (I) wherein R represents the n-dodecyl radical, the n-tetradecyl radical, the n-hexadecyl radical and the n-octadecyl radical;
from 10% to 24% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-dodecyl radical, the n-tetradecyl radical, the n-hexadecyl radical and the n-octadecyl radical;
from 0% to 1% by weight of glucose.

According to a particular aspect, one subject of the process is the preparation of a composition (C) having a color of less than or equal to 1.5 VCS, comprising, for 100% of its weight:
from 75% to 90% by weight of a mixture of alcohols of formula (I) wherein R represents the n-eicosyl radical and the n-docosyl radical;
from 10% to 24% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-eicosyl radical and the n-docosyl radical;
from 0% to 1% by weight of glucose.

According to a particular aspect, one subject of the process is the preparation of a composition (C) having a color of less than or equal to 1.5 VCS, comprising, for 100% of its weight:
from 75% to 90% by weight of a mixture of alcohols of formula (I) wherein R represents the n-dodecyl radical, the n-tetradecyl radical, the n-hexadecyl radical, the n-eicosyl radical and the n-docosyl radical;
from 10% to 24% by weight of at least one composition (C1) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-dodecyl radical, the n-tetradecyl radical, the n-hexadecyl radical, the n-eicosyl radical and the n-docosyl radical;
from 0% to 1% by weight of glucose.

According to a particular aspect, one subject of the process is the preparation of a composition (C) having a color of less than or equal to 1.5 VCS, comprising, for 100% of its weight:
from 70% to 90% by weight of a mixture of alcohols of formula (I) wherein R represents the n-dodecyl radical, the n-tetradecyl radical, the n-hexadecyl radical, the n-eicosyl radical and the n-docosyl radical;
from 10% to 29% by weight of a composition (C1) represented by formula (II) wherein G represents the xylosyl or α,β-D-xylopyranosyl radical, obtained from the removal of the hemiacetal hydroxyl group of α,β-D-xylopyranose, x represents a decimal number of greater than or equal to 1.05 and less than or equal to 2.0, R represents the 2-octyldodecyl radical;
from 0% to 1% by weight of xylose.

According to a particular aspect, the basic agent (Ab) present in the aqueous solution is potassium carbonate of formula (IVa) wherein X represents the potassium atom and n is equal to 2.

According to a particular aspect, the basic agent (Ab) present in the aqueous solution is sodium hydrogen carbonate of formula (IVb) wherein Y represents a sodium atom and m is equal to 1.

According to a particular aspect, the acid catalyst (CA) is selected from the members of the group consisting of sulfuric acid, phosphoric acid, hypophosphorous acid, methanesulfonic acid and p-toluenesulfonic acid.

### EXAMPLES

A—Comparison of the effect of the neutralizing agent on the color of a composition of fatty alcohols and alkyl polyglucosides when the neutralizing basic agent used is a carbonate according to the invention or sodium hydroxide (comparative neutralizing agent).

Comparisons were made between a carbonate and sodium hydroxide as neutralizing agent. To this end, glycosylation reactions were carried out proceeding from crystalline glucose and various fatty alcohols, either in the form of cuts or in pure form: C16/18 cetearyl cut, C20/22 arachidyl/behenyl cut, 1-dodecanol (C12 alcohol).

Table 1 below collates the neutralization results involving sodium carbonate Na₂CO₃ on the one hand (neutralizer of the process according to the invention) and sodium hydroxide NaOH on the other hand (neutralizer of the comparative process).

### 1—Examples According to the Invention

### Example 1.1 (Cut of 16/18 Alcohols and Na₂CO₃ as Neutralizing Agent) According to the Invention

### Step 1: Glycosylation Reaction:

967.4 g of cetearyl alcohol (C16/18) are charged to a reactor equipped with a mechanical stirrer and a vacuum distillation setup. The alcohol is melted at 85° C., stirred and sparged with nitrogen. The medium is placed under vacuum at pressures of less than 50 Torr. An amount of anhydrous glucose in powder form is added such that the molar ratio of fatty alcohols to glucose is 6/1. The medium is inerted under nitrogen. To start the etherification reaction, 0.9 g of a 50% aqueous solution of H₃PO₂ and then 1.1 g of a 98% aqueous solution of H₂SO₄ are added and the temperature is increased and maintained at 105° C. The reaction is continued for a duration of 5 hours 45 minutes.

### Step 2: Neutralization of the Reaction Medium:

The medium is subsequently cooled to 80° C. at atmospheric pressure, then neutralized by introducing 4.61 g of a 25% aqueous solution of Na₂CO₃. The product is subsequently introduced into a glass vial and placed in an oven at 80° C. for 24 hours in order to settle out the residual glucose. The product (upper phase) is recovered and referenced (Composition 1).

### Analyses:

the pH of a 5 wt % dispersion in water of Composition 1 is 5.5, and
the color measurement of Composition 1 is 0.7 VCS.

### Example 1.2 (Cut of 20/22 Alcohols and Na₂CO₃ as Neutralizing Agent)

### Step 1: Glycosylation Reaction:

240.5 g of a mixture of arachidyl alcohol (C20 alcohol) and behenyl/arachidyl alcohol (C220/22 alcohol), in a weight ratio of C20 alcohol to C22 alcohol of 70/30, are charged to a reactor equipped with a mechanical stirrer and a vacuum distillation setup. The alcohol mixture is melted at 85° C., stirred and sparged with nitrogen. The medium is placed under vacuum at pressures of less than 50 Torr. 24.6 g of anhydrous glucose dextrose in powder form are added. The medium is inerted under nitrogen. To start the etherification reaction, 0.2 g of a 50% aqueous solution of H₃PO₂ and then 0.4 g of a 98% aqueous solution of H₂SO₄ are added and the temperature is increased and maintained at 105° C. The reaction is continued for 4 hours 30 minutes.

### Step 2: Neutralization of the Reaction Medium:

The medium is subsequently cooled to 80° C. at atmospheric pressure, then neutralized by introducing 3.3 g of a 10% aqueous solution of Na₂CO₃. The product has a pH of 5.6 and a color of 0.3 VCS. The product is subsequently introduced into a glass vial and placed in an oven at 80° C. for 24 hours in order to settle out the residual glucose dextrose. The product (upper phase) has a pH of 4.8 and a color of 0.3 VCS. A finishing step is carried out at 80° C. by introducing 0.19 g of a 10% aqueous solution of Na₂CO₃ in order to obtain a final product (Composition 2).

### Analyses:

the pH of a 5 wt % dispersion in water of Composition 2 is 7.0, and
the color measurement of Composition 2 is 0.6 VCS.

### Example 1.3 (C12 Alcohol and Na₂CO₃ as Neutralizing Agent)

The procedure of example 1.1 is reproduced, substituting the 967.4 g of cetearyl alcohol for 414.5 grams of 1-dodecanol and using an amount of anhydrous glucose in powder form such that the molar ratio of 1-dodecanol to glucose is 6/1.

The product referenced (Composition 3) is thus obtained.

### Step 1: Glycosylation Reaction:

131.3 g of 1-dodecanol (C12 alcohol) are charged to a reactor equipped with a mechanical stirrer and a vacuum distillation setup. The alcohol is placed at 85° C., stirred and sparged with nitrogen. The medium is placed under vacuum at 30 Torr. 18.3 g of anhydrous glucose in powder form are added. The medium is inerted under nitrogen. To start the etherification reaction, 0.14 g of a 50% aqueous solution of H₃PO₂ and then 0.23 g of a 98% aqueous solution of H₂SO₄ are added and the temperature is increased and maintained at 105° C. The reaction is continued for 5 hours.

### Step 2: Neutralization of the Reaction Medium:

The medium is subsequently cooled to 67° C. at atmospheric pressure, then neutralized by introducing 0.29 g of a 25% aqueous solution of Na₂CO₃ with stirring. The product is filtered on filter paper (˜10 μm) in order to remove the residual glucose to obtain the product referenced (Composition 3).

### Analyses:

the pH of a 5 wt % dispersion in water of Composition 3 is 6.1, and
the color measurement of Composition 3 is 1.5 VCS.

### Example 1.4 (Cut of 16/18 Alcohols and Na₂CO₃ as Neutralizing Agent)

The procedure of example 1.1 is reproduced, substituting the 4.61 grams of a 25% aqueous solution of Na₂CO₃ for the suitable weight of said 25% solution of Na₂CO₃ so as to obtain a pH of 7.4 for a 5% dispersion of the final composition (Composition 4) thus obtained.

### Analyses:

the pH of a 5 wt % dispersion in water of Composition 4 is 7.4, and
the color measurement of Composition 4 is 1.0 VCS.

### Example 1.5 (Cut of 16/18 Alcohols and Na₂CO₃ as Neutralizing Agent)

The procedure of example 1.1 is reproduced, substituting the 4.61 grams of a 25% aqueous solution of Na₂CO₃ for the suitable weight of said 25% solution of Na₂CO₃ so as to obtain a pH of 8.2 for a 5% dispersion of the final composition (Composition 5) thus obtained. The product referenced (Composition 5) is thus obtained.

### Analyses:

the pH of a 5 wt % dispersion in water of Composition 5 is 8.2, and
the color measurement of Composition 5 is 2.3 VCS.

### 2—Comparative Examples

### Example 2.1 (Cut of 16/18 Alcohols and NaOH as Neutralizing Agent)

### Step 1: Glycosylation Reaction:

240.9 g of cetearyl alcohol (C-16/18) are charged to a reactor equipped with a mechanical stirrer and a vacuum distillation setup. The alcohol is melted at 85° C., stirred and sparged with nitrogen. The medium is placed under vacuum at pressures of less than 50 Torr. 31.6 g of anhydrous glucose in powder form are added. The medium is inerted under nitrogen. To start the etherification reaction, 0.22 g of a 50% aqueous solution of H₃PO₂ and then 0.28 g of a 98% aqueous solution of H₂SO₄ are added and the temperature is increased and maintained at 105° C. The reaction is continued for 5 hours 45 minutes.

### Step 2: Neutralization of the Reaction Medium:

The medium is subsequently cooled to 72° C. at atmospheric pressure, then neutralized by introducing 0.73 g of a 25% aqueous solution of NaOH with stirring. The product is subsequently introduced into a glass vial and placed in an oven at 80° C. for 24 hours in order to settle out the residual glucose. The product (upper phase) is recovered (Comparative Composition 1).

### Analyses:

the pH of a 5 wt % dispersion in water of Comparative Composition 1 is 6.1, and
the color measurement of Comparative Composition 1 is 4.1 VCS.

### Example 2.2 (Cut of 20/22 Alcohols and NaOH as Neutralizing Agent)

The procedure of example 2.1 is reproduced, substituting the 240.9 g of cetearyl alcohol for 59.9 grams of a mixture of arachidyl alcohol (C20) and behenyl alcohol (C22) in a (C20 alcohol/C22 alcohol) weight ratio of 70/30, and using an amount of anhydrous glucose in powder form such that the molar ratio of fatty alcohols to glucose is 6/1.

The product referenced (Comparative Composition 2) is thus obtained.

### Analyses:

the pH of a 5 wt % dispersion in water of Comparative Composition 2 is 5.7, and
the color measurement of Comparative Composition 2 is 6.3 VCS.

### Example 2.3 (C12 Alcohol and NaOH as Neutralizing Agent)

### Step 1: Glycosylation Reaction:

131.3 g of 1-dodecanol (C12) are charged to a reactor equipped with a mechanical stirrer and a vacuum distillation setup. The 1-dodecanol is introduced at 85° C., stirred and sparged with nitrogen. The medium is placed under vacuum at 30 Torr. 18.3 g of anhydrous glucose in powder form are added. The medium is inerted under nitrogen. To start the etherification reaction, 0.14 g of a 50% aqueous solution of H₃PO₂ and then 0.23 g of a 98% aqueous solution of H₂SO₄ are added and the temperature is increased and maintained at 105° C. The reaction is continued for 5 hours.

### Step 2: Neutralization of the Reaction Medium:

The medium is subsequently cooled to 67° C. at atmospheric pressure, then neutralized by introducing 0.29 g of a 25% aqueous solution of NaOH with stirring. The product is filtered on filter paper (˜10 μm) in order to remove the residual glucose and to obtain the final product referenced (Comparative Composition 3).

### Analyses:

the pH of a 5 wt % dispersion in water of Comparative Composition 3 is 6.0, and
the color measurement of Comparative Composition 3 is 1.8 VCS.

TABLE 1

| | Comparative | Composition | Comparative | Composition | Comparative | Composition |
|---|---|---|---|---|---|---|
| Reference | composition 1 | 1 | composition 2 | 2 | composition 3 | 3 |
| APG sugar | Glucose | | | | | |
| APG alkyl | C-16/18 | | C-20/22 | | C-12 | |
| chain | | | | | | |
| Neutralizing | NaOH | Na₂CO₃ | NaOH | Na₂CO₃ | NaOH | Na₂CO₃ |
| basic agent | | | | | | |
| pH 5% | 6.1 | 5.5 | 5.7 | 7.0 | 6.0 | 6.1 |
| Color (VCS) | 4.1 | 0.7 | 6.3 | 0.6 | 1.8 | 1.5 |

### Analyses and Observations

Regardless of the length of fatty chain studied (from C12 to C22), the use of a solution of NaOH as basic agent for neutralizing the reaction medium, to achieve pH values of a 5 wt % dispersion in water of greater than or equal to 5.5 of said reaction medium, strongly colors the compositions comprising residual fatty alcohols and the alkyl polyglucosides formed. Indeed, the color measured for these tests fluctuates between 1.8 and 6.3 VCS after filtration of the residual sugar. In comparison, when the neutralization step is conducted using a solution of Na₂CO₃, to achieve pH values of a 5 wt % dispersion in water of greater than or equal to 5.5 of said reaction medium, it is observed that the colors of the compositions comprising residual fatty alcohols and the alkyl polyglucosides formed have values of less than or equal to 1.5 VCS.
B—Influence of the pH of a 5 wt % dispersion in water of a composition of fatty alcohols and alkyl polyglucosides on the color of the final product, when the neutralization phase is carried out with a carbonate

Three experiments were carried out by implementing the general process presented above. These three tests (example 1.1, example 1.4 and example 1.5) describe the preparation of compositions comprising cetearyl alcohol and alkyl polyglucosides on linear C16 and C18 chains, said compositions (Composition 1, Composition 4 and Composition 5) having been prepared by implementing the process according to the invention and being characterized by different pH values of the 5 wt % dispersion of each composition (cf table 2).

TABLE 2

| Test | (Composition 1) | (Composition 4) | (Composition 5) |
|---|---|---|---|
| pH (5%) | 6.4 | 7.4 | 8.2 |
| Color (VCS) | 0.7 | 1.0 | 2.3 |

### Observations and Analyses

The results obtained show that, when the pH of a 5 wt % dispersion of the composition prepared is between 5.5 and 7.5, the process according to the invention makes it possible to achieve a color value of less than or equal to 1.5 VCS. In contrast, when the pH of a 5 wt % dispersion of the composition prepared is 8.2, the color of the composition obtained is greater than the maximum desired color of 1.5 VCS.

1. A process for preparing a composition (C) having a color of less than or equal to 1.5 VCS, comprising, for 100% of its weight:
i) an amount of greater than or equal to 40% by weight and less than or equal to 95% by weight of an alcohol of formula (I):
R—OH (I), wherein R represents a linear or branched, saturated or unsaturated hydrocarbon radical, which may contain at least one hydroxyl function, and containing from 12 to 22 carbon atoms or a mixture of alcohols of formula (I);
ii) an amount of greater than or equal to 5% by weight and less than or equal to 60% by weight of a composition (C1) represented by formula (II):
R—O-(G)x-H (II), wherein the residue G represents the residue of a reducing sugar, R represents a radical as defined in formula (I) and x, which indicates the average degree of polymerization of the residue G represents a decimal number greater than 1.05 and less than or equal to 2.5, or a mixture of compositions (C1) of formula (II);
it being understood that the sum of the weight proportions of the compounds of formulae (I) and (II) in composition (C) is equal to 100% by weight;
said process successively comprising:
a) a step a) of glycosylation, consisting of a reaction between at least one alcohol of formula (I) and at least one reducing sugar of formula (III): H—O-(G)-H (III), in the presence of at least one acid catalyst (CA), at a temperature of greater than or equal to 100° C. and less than or equal to 120° C., the at least one acid catalyst (CA) being selected from members of the group consisting of sulfuric acid, hydrochloric acid, phosphoric acid, nitric acid, hypophosphorous acid, methanesulfonic acid, para-toluenesulfonic acid, trifluoromethanesulfonic acid and acid ion-exchange resins,
b) a step b) of neutralizing the reaction medium from step a) with an aqueous solution comprising a basic agent (Ab) selected from the members of the group consisting of:
carbonates of formula (IVa):
XnCO₃ (IVa), wherein X represents a sodium or potassium atom and n is an integer equal to 2, or X represents a calcium atom or a magnesium atom and n is an integer equal to 1,
or
hydrogen carbonates of formula (IVb):
Y(HCO₃)m (IVb), wherein Y represents a sodium or potassium atom and m is an integer equal to 1, or Y represents a calcium atom or a magnesium atom and m is an integer equal to 2,
the neutralization being carried out so as to obtain a reaction medium for which the 5 wt % dispersion of said reaction medium in water has a pH of between 5.5 and 7.5,
c) a step c) of removing the sugar of formula (III), which did not react in step a), from the neutralized reaction medium, and
d) a step d) of recovering at least one composition (C) having a color of less than or equal to 1.5 VCS.

2. The process as claimed in claim 1, characterized in that said composition (C1) consists of a mixture of compounds represented by formulae (II1), (II2), (II3), (II4) and (II5):
R—O-(G)1-H (II1),
R—O-(G)2-H (II2),
R—O-(G)3-H (II3),
R—O-(G)4-H (II4),
R—O-(G)5-H (II5),
in the respective molar proportions a1, a2, a3, a4 and a5, such that:
the sum: a1+a2+a3+a4+a5 is equal to 1, and
the sum a1+2a2+3a3+4a4+5a5 is equal to x.

3. The process as claimed in claim 1, characterized in that the composition (C) comprises an amount of less than or equal to 2% by weight of the reducing sugar of formula (III):
H—O-(G)-H (III);
it being understood that the sum of the weight proportions of the compounds of formulae (I), (II) and (III) in composition (C) is equal to 100% by weight.

4. The process as claimed in claim 1, characterized in that the basic agent (Ab) present in the aqueous solution is sodium carbonate of formula (IVa) wherein X is a sodium atom and n is equal to 2.

5. The process as claimed in claim 1, characterized in that it comprises, after step c), an additional step of adjusting the pH of a 5 wt % dispersion of the reaction medium in water by adding an aqueous solution comprising the basic agent (Ab) as defined above, so as to obtain a value of said pH of between 5.5 and 7.5.

6. The process as claimed in claim 1, characterized in that the reducing sugar of formula (III) selected for the glycosylation of step a) is selected from the members of the group consisting of glucose, xylose, arabinose and rhamnose.

7. The process as claimed in claim 1, characterized in that step c) of removing the reducing sugar of formula (III) is carried out by filtration, centrifugation or settling.

8. The process as claimed in claim 1, characterized in that, in step b), the aqueous solution of basic agent (Ab) comprises between 10% and 40% by weight of said basic agent (Ab).

9. The process as claimed in claim 1, characterized in that step a) comprises the following successive substeps:
i) introducing an alcohol of formula (I) or a mixture of alcohols of formula (I) into a reactor (Re) equipped with a mechanical stirrer and a vacuum device;
ii) heating the alcohol of formula (I) to a temperature of between 80° C. and 90° C. under mechanical stirring;
iii) charging the reducing sugar of formula (III) into the reactor (Re);
iv) introducing at least one acid catalyst into the reactor (Re);
v) heating, under partial vacuum, the reaction medium from substep iv) present in the reactor (Re) to a temperature of between 100° C. and 110° C. for the duration of the reaction, and
vi) cooling the medium from substep v) to a temperature of between 70° C. and 80° C.

10. The process as claimed in claim 9, characterized in that, in formula (I) and/or in formula (II), the radical R is selected from the following radicals: lauryl (or n-dodecyl), myristyl (or n-tetradecyl), n-pentadecyl, cetyl (or n-hexadecyl), n-heptadecyl, stearyl (or n-octadecyl), palmitoleyl (or 9-hexadecenyl), oleyl (or 9-octadecenyl), linoleyl (9,12-octadecadienyl), linolenyl (or 6,9,12-octadecatrienyl), n-nonadecyl, arachidyl (or n-eicosyl), behenyl (or n-docosyl), erucyl (13-docosenyl), or 12-hydroxystearyl.

11. The process as claimed in claim 9, characterized in that, in formula (I) and/or in formula (II), the radical R is selected from the following radicals: 2-hexyloctyl, 2-hexyldecyl, 2-hexyldodecyl, 2-octyldecyl, 2-octyldodecyl, 2-decyltetradecyl, isostearyl (or 16-methylheptadecyl) or isomyristyl (or 13-methyltridecyl).

12. The process as claimed in claim 9, characterized in that, during substeps ii) to iv), the reactor (Re) is inerted under nitrogen.

13. The process as claimed in claim 9, characterized in that it comprises, between substeps ii) and iii), a vacuum substep, preferably at a pressure of less than or equal to 50 millibar.

14. The process as claimed in claim 9, characterized in that step vi) is carried out at atmospheric pressure.
